(19) United States
(12) Patent Application Publication
(10) Pub. No.: US 2020/0113812 A1
(43) Pub. Date: Apr. 16, 2020

(54) WATER SWELLABLE CROSSLINKED ELASTOMER AND METHOD OF PREPARATION

(71) Applicant: Momentive Performance Materials Inc., Waterford, NY (US)

(72) Inventors: Amar Pawar, Briarcliff Manor, NY (US); Benjamin Falk, Yorktown Heights, NY (US); Mukesh Kumar, New City, NY (US)

(21) Appl. No.: 16/157,566

(22) Filed: Oct. 11, 2018

(51) Int. Cl. A61K 8/896; C08F 220/06; A61Q 19/08; C08F 283/12; A61K 8/81

(52) U.S. Cl. CPC A61K 8/896; C08F 220/06; A61K 8/8147; C08F 283/124; A61Q 19/08

(57) ABSTRACT

A water swellable cross-linked elastomer and method of preparation is provided. The elastomer can advantageously be included in skin treatment compositions. Swellable elastomers in accordance with the invention are particularly well suited for inclusion in skin tightening cosmetic products. The elastomers can be formulated to form a film. When applied to skin, an elastomer in accordance with preferred embodiments of the invention can adhere to the skin and shrink as it dries. This can provide a skin tightening, lifting and wrinkle smoothening effect upon drying. This effect can temporarily mask the appearance of wrinkles.

### FIELD OF THE INVENTION

The invention relates to formulations for skin tightening, sensorial and optical benefits and more particularly to film forming formulations, which upon drying, provide skin tightening and wrinkle smoothening benefits.

### BACKGROUND OF THE INVENTION

There has been a desire to provide formulations for the treatment of the appearance of wrinkles. Various attempts have proposed cosmetic compositions including sodium silicate and/or polyacrylamide/polyacrylate copolymers alone or in admixture with sodium polyacrylate. Other options have proposed different film forming compositions. However, the results have not been fully acceptable.

U.S. Pat. No. 6,139,829 alleges temporary smoothening of wrinkle appearance using ionic polyacrylamide/polyacrylate copolymer alone or in an admixture with sodium polyacrylate. US 2015/0037380 alleges skin smoothening compositions comprising sodium silicate, polyvalent silicate and water. U.S. Pat. No. 7,700,084 alleges a skin tightening composition containing a high molecular weight polysaccharide, polyvinyl alcohol, sorbitol and cellulose. US 2017/0189299 alleges a skin tightening film with a polyvalent silicate thickener. U.S. Pat. No. 8,580,741 alleges a skin tightening composition containing high molecular weight plant protein and glycoprotein. EP 0 180 968 A2 alleges an anti-wrinkle composition containing serum albumin. US 2017/0189299 alleges a skin tightening aqueous film forming composition containing sodium silicate, pullulan, acrylates copolymer, and bentonite. U.S. Pat. No. 6,284,233B1 alleges antiwrinkle compositions comprising a combination of tightening polymers of synthetic and/or natural origin and of dendritic polyesters. WO 2013/076238 describes cosmetic compositions containing acrylic acid homopolymers or copolymers. U.S. Pat. No. 7,687,574 describes acrylate cross linked silicone copolymer networks. U.S. Pat. No. 9,387,161 alleges moisturizing and line blurring compositions containing cross-linked acrylate silicone copolymers. U.S. Pat. No. 9,616,253 describes water absorbing cross-linked silicone copolymers. The entire contents of the above citations are incorporated herein by reference.

An issue with these prior attempts has been the insufficient reduction in the appearance of wrinkles and/or an undesirable skin whitening effect. This whitening can occur when the film dries, flakes off and forms a whitening powder. This can undesirably lead to immediate loss of anti-wrinkle effect of such formulations.

Accordingly, it is desirable to provide an improved composition that overcomes drawbacks of the prior art.

### SUMMARY OF THE INVENTION

Generally speaking, in accordance with the invention, water swellable cross-linked elastomers, compositions containing those elastomers and methods of preparation are provided. The elastomer can advantageously be included in skin treatment formulations. Swellable elastomers in accordance with the invention are particularly well suited for inclusion in skin tightening cosmetic products. The elastomers can be formulated to form a film. When applied to skin in the water swollen condition, elastomers in accordance with preferred embodiments of the invention can adhere to the skin. The film can shrink as it dries and remain adhered to the skin. This can provide a skin tightening, lifting and wrinkle smoothening effect upon drying of the water swollen film. This effect can temporarily mask the appearance of wrinkles.

Cosmetic formulations in accordance with the invention can exhibit high durability of skin tightening, lifting and wrinkle smoothening effect. Lack of durability in current skin tightening cosmetics can be due to failure of dried films to maintain their integrity on the skin surfaces to which they are applied. Such unacceptable films can lead to flaking which lead to whitening of skin due to white residue of flakes. However, when applying compositions in accordance with the invention, at least about 80, even 90% of the film will remain in place, without flaking and leading to whitening, for at least about 2, even 4 hours. Preferred formulations will remain in place at least about 75% by weight for at least six hours. This leads to reduced whitening as compared to skin tightening films that begin to flake in less time. All percentages herein are by weight.

Elastomers in accordance with the invention include cross linked polyacrylates containing at least one phosphate moiety that can provide a skin tightening and/or wrinkle lifting effect as they dry. Elastomers in accordance with the invention can be formed as the cross linked reaction product of a siloxane component, preferably polydimethyl siloxane; an acrylic or methacrylic monomer; and a phosphate or a phosphonate group containing monomer. Elastomers in accordance with the invention preferably are formed as the reaction product of reactants, of which about 2 wt %-50 wt %, more preferably 20 wt %-40 wt % comprise a phosphate or phosphonate monomer. Preferred elastomers include about 0.01%-40% by weight of the phosphate or phosphonate moiety. In more preferred embodiments of the invention, the elastomer includes about 2% to 15% by weight of the phosphate or a phosphonate moiety. (Unless specified otherwise, all percentages herein will be by weight). The elastomer can be provided as a dry powder or in the form of a water swollen gel. The gel will preferably include about 5 to 20 wt % of the elastomer and water.

Water swellable elastomers in accordance with the present invention can be used in a broad range of personal care formulations including, but not limited to skin care, color cosmetics and sun care formulations, such as eye creams, moisturizers, night repair lotions, sun creams, lipsticks, foundations, lotions, and masks.

The elastomer can be formed as a reaction product from a monomer with the general formulae (V) to (VIII) and/or the cross linker of formula (IX):

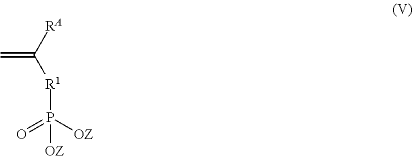

wherein R¹ is a C1-C60 alkyl chain, which can be linear, branched or cyclic optionally containing a heteroatom (O, N, S); Z is H or an alkali or alkaline metal ion or an ammonium ion; and R^{A} is H or CH₃.

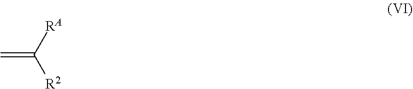

wherein R² is a C1-C60 alkyl chain which can be linear, branched or cyclic optionally containing a heteroatom (O, N, S); and R^{A} is H or CH₃.

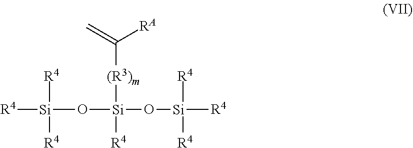

wherein m is 0 or 1; R³ is a C1-C60 alkyl chain which can be linear, branched or cyclic optionally containing a heteroatom (O, N, S); R^{A} is H or CH₃; and each R⁴ is independently a monovalent hydrocarbon radical independently having from 1 to 20 carbon atoms; or

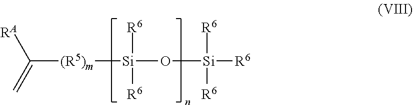

wherein m is 0 or 1; R⁵ is a C1-C60 alkyl chain which can be linear, branched or cyclic optionally containing a heteroatom (O, N, S); R^{A} is H or CH₃; and each R⁶ is independently a monovalent hydrocarbon radical having from 1 to 20 carbon atoms; and n=3 to 500; The cross-linker of general formula (IX)

R⁷(R⁸)ₐ (IX)
wherein a≥2;
R⁷ is a bivalent group connected to R⁸ and R⁷ can be
i) a bivalent hydrocarbon radical having from 1-20 carbon atoms, optionally containing heteroatom (O, S, N);
ii) a bivalent phosphoric acid formed by the bonding of O and an alkyl radical;
iii) [R⁹R¹⁰SiO]ₙ wherein R⁹ and R¹⁰ are monovalent hydrocarbon radicals having from 1 to 20 carbon atoms, n=3-500; or
iv) MᵤDᵥTₓQ_{y} (M=R¹¹₃SiO_{1/2}, D=R¹²₂SiO_{2/2}, T=R¹³SiO_{3/2}, Q=SiO_{4/2}) wherein R¹¹, R¹², R¹³ are monovalent hydrocarbon radicals having from 1 to 20 carbon atoms; u, v, x, y are independent integers from 0 to 300, preferably 0-100, more preferably 0-50, and most preferably from 0-20, u+v+x+y is subject to limitations ≥1; and
R⁸ is monovalent unsaturated hydrocarbon radical connected to R⁷.

Accordingly, it is an object of the invention to provide an improved elastomer and method of formation and use.

Still other objects of the invention will in part be obvious and will, in part be apparent from the specification and drawings and the scope of the invention will be indicated in the claims.

### BRIEF DESCRIPTION OF THE DRAWINGS

For a fuller understanding of the invention, reference is had to the following description, taken in connection with the accompanying drawings, in which:

FIG. 1 is a graph depicting the lift distance of the examples;

FIG. 2 shows percent elastic recovery of certain examples depicted;

FIG. 3 shows color change in ΔE among the Examples as a measure of lightening; and

FIG. 4 shows Visioscan images of the forearm of a test subject, before and after drying the test solutions on the subject's forearm for 15 minutes.

### DETAILED DESCRIPTION OF THE PREFERRED EMBODIMENTS OF THE INVENTION

In the specification and claims herein, the following terms and expressions are to be understood as indicated.

The singular forms “a,” “an,” and “the” include the plural, and reference to a particular numerical value includes at least that particular value, unless the context clearly dictates otherwise.

Other than in the working examples or where otherwise indicated, all numbers expressing amounts of materials, reaction conditions, time durations, quantified properties of materials, and so forth, stated in the specification and claims are to be understood as being modified in all instances by the term “about”.

All methods described herein may be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed.

No language in the specification should be construed as indicating any non-claimed element as essential to the practice of the invention.

The terms, “comprising,” “including,” “containing,” “characterized by,” and grammatical equivalents thereof are inclusive or open-ended terms that do not exclude additional, unrecited elements or method steps, but will also be understood to include the more restrictive terms “consisting of” and “consisting essentially of.”

It will be understood that any numerical range recited herein includes all sub-ranges within that range and any combination of the various endpoints of such ranges or sub-ranges.

As used herein, integer values of stoichiometric subscripts refer to molecular species and non-integer values of stoichiometric subscripts refer to a mixture of molecular species on a molecular weight average basis, a number average basis or a mole fraction basis.

It will be further understood that any compound, material or substance which is expressly or implicitly disclosed in the specification and/or recited in a claim as belonging to a group of structurally, compositionally and/or functionally related compounds, materials or substances includes individual representatives of the group and all combinations thereof.

The term “adjuvant” means any composition, material or substance which increases the efficacy of the active material to which it is added.

The expression “hydrocarbon group” or “hydrocarbon radical” means any hydrocarbon from which one or more hydrogen atoms has been removed and is inclusive of alkyl, alkenyl, alkynyl, cyclic alkyl, cyclic alkenyl, cyclic alkynyl, aryl, aralkyl and arenyl groups and is inclusive of hydrocarbon groups containing at least one heteroatom.

The term “alkyl” means any monovalent, saturated straight, branched or cyclic hydrocarbon group; the term “alkenyl” means any monovalent straight, branched, or cyclic hydrocarbon group containing one or more carbon-carbon double bonds where the site of attachment of the group can be either at a carbon-carbon double bond or elsewhere therein; and, the term “alkynyl” means any monovalent straight, branched, or cyclic hydrocarbon group containing one or more carbon-carbon triple bonds and, optionally, one or more carbon-carbon double bonds, where the site of attachment of the group can be either at a carbon-carbon triple bond, a carbon-carbon double bond or elsewhere therein. Examples of alkyls include methyl, ethyl, propyl and isobutyl. Examples of alkenyls include vinyl, propenyl, allyl, methallyl, ethylidenyl norbornane, ethylidene norbornyl, ethylidenyl norbornene and ethylidene norbornenyl. Examples of alkynyls include acetylenyl, propargyl and methylacetylenyl.

The present invention relates to water swellable crosslinked elastomers in powder and gel form; formulations including the elastomers, and a processes of forming the same and its application for skin tightening, wrinkle smoothening and optical effects. A cosmetically acceptable composition, in accordance with the invention can include a water swellable elastomer formulated to provide tightening to a keratinous substrate to which it is applied. The elastomer should be formed from monomers that include at least one acrylic or methacrylic moiety, at least one cross linking moiety and at least one phosphate moiety.

The cosmetically acceptable compositions can include a moiety from a vinyl terminated polydimethylsiloxane and an acrylic or methacrylic polyether siloxane copolymer. The vinyl groups of the elastomer can be terminal, pendant or a mixture of terminal and pendant groups. As used herein, the term polyacrylate will be used to refer to polymers of both acrylates and methacrylates, unless specified differently. Elastomers of the composition can comprise a moiety from 2-hydroxyethyl methacrylate phosphate. The elastomers can comprise a moiety from a vinyl terminated polydimethylsiloxane with the average structure CH₂═CHSi(CH₃)₂O[Si(CH₃)₂O]ₙSi(CH₃)₂CH═CH₂, wherein n is about 100 to 2000. In more preferred embodiments of the invention, n is about 600 to 1000.

Compositions in accordance with the invention can include an elastomer comprising a moiety from tripropyleneglycoimethacrylate monophosphate, hydroxyethylmethacylate monophosphate and other phosphates. Elastomers in accordance with the invention are preferably formed as the reaction product of components, about 2 wt %-50 wt %, more preferably 20 wt %-40 wt % of which comprise a phosphate or phosphonate monomer. Preferred elastomers preferably include about 0.01%-40% by weight of the phosphate or phosphonate moiety. In more preferred embodiments of the invention, the phosphate or phosphonate moiety comprises 2% to 15% by weight of the elastomer composition.

Compositions of the invention can be effective to temporarily reduce the appearance of skin wrinkles of a user. Such compositions can be formulated to form a continuous film on and adhere to a keratinous substrate, wherein, when undisturbed, at least about 75 wt % of the film, more preferably about 90 wt % will remain as a film on the substrate, without flaking off after four, preferably six hours.

Elastomers used in compositions in accordance with the invention can be formed from a reaction comprising a siloxane polymer. The siloxane is preferably a polydimethyl siloxane. Examples include polydimethyl siloxanes. Preferably, the siloxane comprises about 100 to 2000, more preferably 300 to 500 siloxane groups.

The carboxylic acid group or carboxylate ion of an acrylate polymer is capable of hydrogen bonding with water and other hydroxylic solvents. This can cause the elastomer to swell when combined with water. The amount of crosslinking present in the crosslinked network may be characterized with respect to the degree of swelling exhibited by the network in water. The elastomer can swell from its original volume to a swollen volume that is a factor of from 1.01 to 5000, preferably from 2 to 1000, and more preferably from 5 to 500, times its original volume. The original volume of the network can be determined, for example, by extracting or evaporating all of the water from the swollen elastomer to leave the original volume, that is, the un-swollen volume of the elastomer in the absence of water.

Compositions in accordance with the invention can be formulated as a personal care product, and can include, but are not limited to an eye cream, eye lotions, deodorants, antiperspirants, antiperspirant/deodorants, shaving products, skin lotions, moisturizers, toners, bath products, cleansing products, hair care products, manicure products, protective creams, or color cosmetics. These compositions can contain a thickener, a particulate, a plasticizer, a surfactant (nonionic, cationic, anionic, and zwitterionic) and/or a skin care active agent or combinations thereof.

A water swellable elastomer suitable for the invention can be formed as the reaction product of reactant components, comprising: a monomer component having 3 to 60 carbon atoms; an acrylate or methacrylate component having 3 to 60 carbon atoms and acrylic or methacrylic functionality; a phosphate component having 3 to 60 carbon atoms; and a siloxane component having a siloxane moiety. The monomer component, acrylate component, phosphate component and siloxane component may be the same or different components and comprise an unsaturated straight, branched or cyclical carbon chain, which may optionally include heteroatoms.

Compositions of the present invention may optionally contain up to 90 parts by weight of one or more particulate materials. Particulate materials suitable for use herein can be but are not limited to inorganic and organic colored and uncolored pigments, optical enhancer particles, organic and inorganic powders and interference pigments. These can comprise aluminum, barium or calcium salts or lakes. A lake is a pigment that is extended or reduced with a solid diluent or an organic pigment that is prepared by the precipitation of a water-soluble dye on an adsorptive surface, such as aluminum hydrate. Other colors and pigments can also be included in the compositions. Other particulates such as silica, nylon microspheres, polyurethane beads, PMMA beads, polymethylsilsesquioxane microspheres can also be included.

Composition of the present invention may optionally contain one or more known or conventional cosmetically-acceptable organic film former. Examples of useful film-forming agents include but are not limited to natural waxes, polymers such as polyethylene polymers, copolymers of PVP, ethylene vinyl acetate, dimethicone gum, resins such as shellac, polyterpenes, silicone resins, and the like.

Compositions of the present invention can be incorporated into a carrier, specifically a volatile carrier which quickly volatilizes after application. The volatile carriers of the present invention are selected from the group consisting of volatile hydrocarbons, volatile silicones and mixtures thereof. Hydrocarbon oils useful in the present invention include those having boiling points in the range of 60-260° C., more preferably hydrocarbon oils having from about C₈ to about C₂₀ chain lengths, most preferably isoparaffins. Most preferred are selected from the group consisting of isododecane, isohexadecane, isoeocosane, 2,2,4-trimethylpentane, 2,3-dimethylhexane and mixtures thereof. Preferred volatile silicone fluids include cyclomethicones having 3, 4 and 5 membered ring structures corresponding to the formula (R₂SiO)ₓ, where x is from about 3 to about 6 or linear methicones corresponding to the formula (R₃SiO(R₂SiO)ₓSiR₃ where x is from about 0 to about 6; and R is H or a monovalent alkyl radical.

A thickening polymer may be useful in the present invention. The expression “thickening polymer” shall be understood for the purposes of the present invention to mean a polymer having, in solution or in dispersion containing 1% by weight of active material in water or in ethanol at 25° C., a viscosity greater than 0.2 poise at a shear rate of 1 s-I. The viscosity can be measured with a HAAKE RS600 viscometer from THERMO ELECTRON. This viscometer is a controlled-stress viscometer with cone-plate geometry (for example, having a diameter of 60 mm and an angle of 10). Examples of thickeners include but are not limited to: associative thickeners; crosslinked acrylic acid homopolymers; crosslinked copolymers of (meth)acrylic acid and of (C₁-C₆) alkyl acrylate; nonionic homopolymers and copolymers containing ethylenically unsaturated monomers of ester and/or amide type; ammonium acrylate homopolymers or copolymers of ammonium acrylate and of acrylamide; (meth)acrylamido(C1-C₄) alkylsulphonic acid homopolymers and copolymers; crosslinked methacryloyl (C₁-C₄) alkyltri (C₁-C₄) alkylarnmonium homopolymers and copolymers. Particulate thickeners may also be used. Also, naturally derived polymers and polymers produced by fermentations may be used such as polysaccharide gums, xanthan gum, pullulan gum, sclerotium gum, carrageenan gum, locust bean gum, alginate, gellan gum, cellulose, carboxymethylcellulose, hydroxyethylcellulose, pectins, starch, chitosan, gelatin and their combination.

Useful additives include pH adjusters/buffering agents and chelating agents such as ammonium hydroxide, sodium hydroxide, potassium hydroxide, C₁₂-C₁₅ alkyl benzoate, citric acid, glycolic acid, lactic acid, sodium citrate, triethanolamine, trolamine, disodium EDTA, edetate disodium, pentasodium pentetate, tetrasodium EDTA, trisodium EDTA.

Skin protectants and humectants may be used in the compositions of the present invention such as dimethicone, petrolatum, glycerin, ammonium lactate, lanolin, methyl gluceth-20, PEG-20, sorbitol, 1,2,6 hexanetriol, butylene glycol, dipropylene glycol, glycerin, hexylene glycol, panthenol, phytantriol, panthenol, propylene glycol, sodium PCA, sorbitol, triethylene glycol, polyglyceryl sorbitol, glucose, fructose, polydextrose, potassium pea, urea, hydrogenated honey, hyaluronic acid, inositol, hexanediol beeswax, hexanetriol beeswax, hydrolyzed elastin, hydrolyzed collagen, hydrolyzed silk, hydrolyzed keratin, erythritol, capryl glycol, and the like. Compositions in accordance with the invention may also contain skin care actives, such as vitamin A, Vitamin C, Vitamin E, botanical extracts, skin brightening agents and mixtures thereof. Compositions of the invention may also contain plasticizers such as polyols, mono-, di-, oligosaccharides. Compositions can also contain polyol plasticizers selected from glycerol, ethylene glycol, diethylene glycol, propylene glycol, sorbitol, urea, triethanolamine, and mixtures thereof.

Preferred elastomers in accordance with the invention include the reaction product of an acrylic or methacrylic monomer component with a source of phosphate functionality. The reactants can also include silicone functional monomers and cross linkers. Elastomer in powder form can easily swell in water and provides water thickening ability. The cross-linked density of the elastomer can be controlled to provide an elastomer with low cross-linked density to form a film upon drying or an elastomer with higher cross-linked density to dry as particulate. Elastomers in accordance with the invention can form a film that adheres to the skin surface. The film will shrink upon drying and can tighten the skin and lift the area of a wrinkle

Surprisingly it was found that elastomers with phosphate functionality (ionic functionality) exhibit enhanced skin tightening and temporary wrinkle smoothening along with silky sensory benefit and enhanced optical properties. Such polymers can have enhanced durability without flaking off and forming powdery residue after longer durations compared to other elastomers.

Swellable elastomers in accordance with the invention can be formulated to form a film. The film will adhere to the skin, dry to a clear film and shrink. As it shrinks, it will thereby provide a skin tightening and wrinkle smoothening effect. This effect can temporarily mask the appearance of wrinkles. The wrinkle smoothening effect relates to how well the film adheres to the skin and how much the film shrinks, and thereby “lifts” the wrinkle upon drying.

If the film begins to detach from the skin and flake off the skin as particles, it can form a white powder. This can create an undesirable appearance, including skin whitening. Films formed from elastomers in accordance with the invention have high durability. Accordingly, they exhibit reduced skin whitening effects as compared to other films.

In one embodiment of the invention, the elastomer is formed from a monomer with at least one unsaturated bond and 3 to 60 carbon atoms, straight chain, branched or cyclical. The monomer can include heteroatoms in the chain. Preferred monomers have acrylic or methacrylic functionality. The elastomer also benefits from a source of phosphate functionality. The phosphate moiety can be part of a straight, branched or cyclical organic molecule with at least one unsaturated bond (PO₄—R²). In another embodiment of the invention, the monomer includes the phosphate functionality (R¹—PO₄). The monomer and phosphate source can be combined with a cross linker. They can also be combined with an acrylate or methacrylate functionalized siloxane (SiO—R³R⁴R⁵). Preferred siloxanes include polydimethyl siloxanes, preferably comprising about 100 to 2000 siloxane groups. R¹ through R⁵ can independently be selected from hydrogen or straight, branched or cyclical carbon chains of 1-60 carbon atoms, which may include heteroatoms in the chain.

In one embodiment of the invention, a monomer used to form said elastomer composition can be represented as:

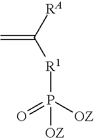

wherein R¹ is a C1-C60 alkyl chain, which can be linear, branched or cyclic optionally containing a heteroatom (O, N, S). Z is H or alkali or alkaline metal ion or an ammonium ion; R^{A} can be H or CH₃. In more preferred embodiments of the invention, R¹ is a C2-C20 alkyl chain, which can be linear, branched or cyclic optionally containing a heteroatom (O, N, S). Preferably, R¹ can be constituted as a single or repetitive unit from a group of (poly)alkylene oxide e.g. (poly)ethylene oxide, (poly)propylene oxide, (poly)butylene oxide, (poly)cyclohexylene oxide or a combination thereof and attached to an O containing group such as carbonyl. More preferably, examples of the structure can be but are not limited to 2-hydroxyethylacrylate phosphate, 2-hydroxyethylmethaerylate phosphate, hydroxypropylmethacrylate phosphate, and bis(2-methacryloxyethyl) phosphate. Even more preferably, representative examples can be depicted as: CH₂═C(CH₃)C(═O)—O—(C₂H₄O)—P(═O)(OH)₂ and CH₂═C(C₃)—C(═O)O—(C₃H₆O)₃−P(═O)(OH)₂. The examples of phosphonate group containing monomers include, but are not limited to diethyl 2-vinylethylphosphonate, n-butylacrylamide phosphonate, (meth)acrylamide phosphonate, Diethyl vinylphosphonate, N-phenyl maleimide phosphonate, styrene phosphonate, dimethyl(methacryloyloxymethyl) phosphonate, diethyl-(4-vinylphenyl)phosphate, diethyl-[2-(diethylphosphinyl)-4-vinylphenyl]phosphate, diphenyl-(4-vinylphenyl)phosphate, diethyl-(4-vinylbenzyl)phosphonate, and diphenyl-(4-vinylbenzyl)phosphonate.

Elastomers in accordance with the invention can be formed from additional monomers, which can be represented as:

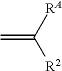

wherein R² is a C1-C60 alkyl chain which can be linear, branched or cyclic optionally containing a heteroatom (O, N, S). R^{A} can be H or CH₃. Preferably, R² is a C1-C20 alkyl chain which can be linear, branched or cyclic optionally containing a heteroatom (O, N, S). More preferably, R² can be an organic or inorganic acid, an ester, or an amide group. Examples of organic acids containing such a group can be but not limited to acrylic acid, itaconic acid and methacrylic acid; examples of ester containing monomers can be but not limited to methyl methacrylate, butylacrylate, ethylamethacrylate, butylmethacrylate, dodecylmethacrylate, hydroxyethylmethacrylate, hydroxypropylmethacrylate; examples of amide group containing monomers can be but are not limited to acrylamide, and methacrylamide, dimethylaminoethyl acrylamide; examples of amine containing monomers can be but are not limited to dimethylaminoethyl acrylate, dimethylaminoethyl methacrylate, p-dimethylaminomethyl styrene; and examples of vinyl monomers can be but are not limited to vinyl pyridine, methyl vinyl pyridine, and vinyl pyrrolidone.

In another embodiment of the invention, the acid group of the acrylic acid is neutralized to form partially neutralized acrylate polymer. Suitable bases for neutralization of polymer can be chosen from a group of alkali or alkaline metal hydroxides, carbonates, bicarbonates, phosphates; aliphatic or aromatic organic amines e.g. primary, secondary or tertiary amines; and tetra-alkyl or tetra-aryl ammonium hydroxides. Suitable examples can be but are not limited to sodium hydroxide, potassium hydroxide, potassium carbonate, sodium carbonate, ammonium carbonate, tributylamine, trimethylamine, triethylamine, pyridine, aniline, ethanolamine, and ammonium hydroxide, tetramethylamonium hydroxide, tetrabutylamonium hydroxide.

In another embodiment of the invention, the polymerization is performed using a radical polymerization imitator. Suitable examples of free radical polymerization initiators can be but are not limited to azo-bis-isobutyrylnitrile, dimethyl 2,2′-azobisisobutyrate, benzyol peroxide, di-tert-butyl peroxide, tert-butyl peroxybenzoate, di-tert-butyl peroxyoxlate, and potassium persulfate.

This monomer can impart siloxane functionality. It can have the acrylic or methacrylic general formula:

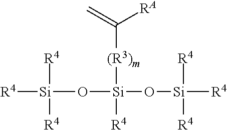

wherein m is 0 or 1; R³ is a C1-C60 alkyl chain which can be linear, branched or cyclic optionally containing a heteroatom (O, N, S); R^{A}═H, CH₃; each R⁴ is independently a monovalent hydrocarbon radical having from 1 to 20 carbon atoms. Preferably, R³ can be represented as a bivalent groups such as (CH₂)ₙC(═O)O, (CH(CH₃))ₙC(═O)O, and (CH₂)ₙC₆H₉(OH)C(═O)O or similar structure thereof. More preferably, these groups are attached to a silicon atom in the siloxane structure by the chemical reaction of epoxy containing siloxane and acrylic acid, methacrylic acid or an acid that contains acrylic or methacrylic moiety.

The monomer can also have the general formula:

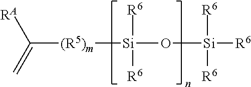

wherein m is 0 or 1; R⁵ is a C1-C60 alkyl chain which can be linear, branched or cyclic optionally containing a heteroatom (O, N, S); R^{A} ═H, CH₃; R⁶ is a monovalent hydrocarbon radical having from 1 to 20 carbon atoms; and n=3-500. Preferably, R⁵ can be represented as a bivalent group such as (CH₂)ₙC(═O)O, (CH(CH₃))C(═O)O, (CH₂)ₙC₆H(OH)C(═O)O. More preferably, these groups are attached to silicon in the said structure by the chemical reaction of epoxy containing siloxane and acrylic acid, methacrylic acid or an acid that contains acrylic or methacrylic moiety.

Elastomeric compositions in accordance with the invention can be made using a cross-linker, which can be represented as:

R₇(R⁸)ₐ
Wherein a≥2;
R⁷ is a bivalent group connected to R⁸ and R⁷ can be
i) a bivalent hydrocarbon radical having from 1-20 carbon atoms, optionally containing heteroatom (O, S, N);
ii) a bivalent phosphoric acid formed by the bonding of O and an alkyl radical;
iii) [R⁹R¹⁰SiO]ₙ wherein R⁹ and R¹⁰ are monovalent hydrocarbon radicals having from 1 to 20 carbon atoms, n=3-500; or
iv) MᵤDᵥTₓQ_{y} (M=R¹¹₃SiO_{1/2}, D=R¹²₂SiO_{2/2}, T=R¹³SiO_{3/2}, Q=SiO_{4/2}) wherein R¹¹, R¹², R¹³ are monovalent hydrocarbon radicals having from 1 to 20 carbon atoms; u, v, x, y are independent integers from 0 to 300, preferably 0-100, more preferably 0-50, and most preferably from 0-20, u+v+x+y is subject to limitations ≥1; and
R⁸ is monovalent unsaturated hydrocarbon radical connected to R⁷.

Examples of cross-linkers can be, but are not limited to di- or triacrylic acid esters of polyols such as ethylene glycol, trimethylol propane triacrylate, glycerine, or polyoxyehtylene glycols. Preferably, these can be diethylene glycol diacrylate, diethylene glycol dimethacrylate, ethylene glycol dimethacrylate, 1,6-hexanediol diacrylate, tetraethylene glycol dimethacrylate, triethylene glycol diacrylate, and bis(2-methacryloxyethyl) phosphate.

Compositions can be formulated, such that after application and drying, at least about 80 to 90% of the film will remain in place, without flaking off, for at least about 2 to 4 hours. About 75% of preferred formulations will remain in place for at least 6 hours. This leads to reduced whitening as compared to skin tightening films that begin to flake off in less time.

In one embodiment of the invention, the elastomer is formed as a reaction product, having phosphate functionality, of a polydimethyl siloxane and an acrylic polymer. Elastomers in accordance with the invention preferably include about 0.01%-40% of a phosphate moiety. In more preferred embodiments of the invention, the phosphate moiety comprises 2% to 10% of the elastomer composition.

The elastomer can be provided as a dry-powder or in the form of a gel. The gel will preferably include about 5 to 20% of the polymer and water.

The water swellable elastomer described in the present invention can be used in broad range of personal care formulations including skin care, color cosmetics and sun care formulations such as eye cream, moisturizers, night repair lotions, sun creams, lipsticks, foundations, and lotions.

### EXAMPLE

The monomers and other raw materials were procured from commercial vendors and used without any further purification: Acrylic acid—Acros Organics (New Jersey, USA); 2-Hydroxyethyl methacrylate—Aldrich (Missouri, USA); Glycerol dimethacrylate—TCI (Oregon, USA); Azobisisobutyronitrile—Aldrich (Missouri, USA); 2-hydroxyethyl methacrylate phosphate—Esstech (PA, USA); Potassium carbonate—Acros Organics (New Jersey, USA); Vinyl terminated polydimethylsiloxane—Momentive Performance Materials (NY, USA).

The following Examples are presented for purposes of illustration only and should not be interpreted as limiting the scope of the invention.

### Synthesis Example 1

Acetone (503.43 g), acrylic acid (53.28 g), 2-hydroxyethyl methacrylate phosphate (27.06 g), potassium carbonate (13.82 g) and azobisisobutyronitrile (AIBN) (0.542 g) were added to a 1 gallon Ross mixer (model DPM-4) and purged with nitrogen. The reaction medium was agitated at 30 rpm and the temperature of the reactor was raised to 60 C. A white precipitate of elastomer material in accordance with the invention formed within 15 to 20 minutes. The reactor was stirred for an additional 3 hours. The stirring was discontinued and the contents were cooled to room temperature. The precipitate was allowed to settle from the solution. The supernatant was decanted and the solid was isolated. The solid was removed and filtered in a Buckner funnel. The resulting cake was washed with 400 g of acetone. The cake was transferred to a glass dish and dried in a vacuum oven at 50° C. overnight. The resulting free flowing powder was white in appearance. The resulting elastomer was comprised of 3.79 wt % phosphate moiety.

### Synthesis Example 2

Acetone (503.42 g), a vinyl terminated polydimethylsiloxane with the average structure CH₂═CHSi(CH₃)₂O[Si(CH₃)₂O]₉₀₀Si(CH₃)₂CH═CH₂ (2.02 g), acrylic acid (53.30 g), 2-hydroxyethyl methacrylate phosphate (27.02 g), potassium carbonate (13.81 g) and azobisisobutyronitrile (0.541 g) were added to a 1 gallon Ross mixer (model DPM-4) and purged with a nitrogen blanket. The reaction medium was agitated at 30 rpm and the temperature of the reactor was raised to 60 C. A white precipitate of elastomer material in accordance with the invention formed within 15 to 20 minutes. The reactor contents were stirred for an additional 3 hours. The stirring was discontinued and the contents were cooled to room temperature. The precipitate was allowed to settle from the solution. The supernatant was decanted and the solid was isolated. The solid was removed and filtered in a Buckner funnel. The resulting cake was washed with 400 g of acetone. The cake was transferred to a glass dish and dried in a vacuum oven at 50° C. overnight. The resulting free flowing powder was white in appearance. The resulting elastomer was comprised, of 3.69 wt % phosphate moiety.

### Synthesis Example 3

Acetone (483.35 g), a vinyl terminated polydimethylsiloxane with the average structure CH₂═CHSi(CH₃)₂O[Si(CH₃)₂O]₉₀₀Si(CH₃)₂CH═CH₂ (2.06 g), an acrylate polyether siloxane copolymer with the average structure of shown below (20.04 g), acrylic acid (10.02 g), tripropyleneglycolmethacrylate monophosphate (70.32 g), potassium carbonate (13.82 g) and azobisisobutyronitrile (0.55 g) were added to a 1 gallon Ross mixer (model DPM-4) and purged with a nitrogen blanket. The reaction medium was agitated at 30 rpm and the temperature of the reactor was raised to 60° C. A white precipitate of elastomer material in accordance with the invention formed within 15 to 20 minutes. The reactor contents were stirred for an additional 3 hours. The stirring was discontinued and the contents were cooled to room temperature. The precipitate material was allowed to settle from the solvent solution. The supernatant was decanted and the solid was isolated. The solid was removed and filtered in a Buckner funnel. The resulting cake was washed with 400 g of acetone. The cake was transferred to a glass dish and dried in the vacuum oven at 500° C. overnight. The resulting free flowing powder was off white to yellow in appearance. The resulting elastomer was comprised of 13.48 wt % phosphate moiety.

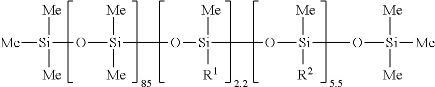

wherein
R¹ ═(CH₂)₂C₆H₉(OH)(OC(═O)CH═CH₂.
R²═(CH₂)₃(OCH₂CH₂)₂₅(OCH(CH₃))₂₉OCH₃

### Synthesis Example 4

Acetone (483.3 g), a vinyl terminated polydimethylsiloxane with the average structure CH₂═CHSi(CH₃)₂O[Si(CH₃)₂O]₉₀₀Si(CH₃)₂CH═CH₂ (2.02 g) depicted above, (20.06 g), acrylic acid (44.28 g), 2-hydroxyethyl methacrylate phosphate (36.02 g), potassium carbonate (13.82 g) and azobisisobutyronitrile (0.541 g) were added to a 1 gallon Ross mixer (model DPM-4) and purged with a nitrogen blanket. The reaction medium was agitated at 30 rpm and the temperature of the reactor was raised to 60° C. A white precipitate of elastomer material in accordance with the invention formed within 15 to 20 minutes. The reactor contents were stirred for an additional 3 hours. The stirring was discontinued and the contents cooled to room temperature. The precipitate material was allowed to settle from the solvent solution. The supernatant was decanted and the solid was isolated. The solid was removed and filtered in a Buckaner funnel. The resulting cake was washed with 400 g of acetone. The cake was transferred to a glass dish and dried in the vacuum oven at 50° C. overnight. The resulting free flowing powder was white in appearance. The resulting elastomer was comprised of 6.56 wt % phosphate moiety.

### Synthetic Example 5

Acetone (503.38 g), a vinyl terminated polydimethylsiloxane with the average structure CH₂═CHSi(CH₃)₂O[Si(CH₃)₂O]₉₀₀Si(CH₃)₂CH═CH₂ (2.03 g) as depicted above, acrylic acid (44.29 g), 2-hydroxyethyl methacrylate phosphate (36.05 g), potassium carbonate (13.82 g) and azobisisobutyronitrile (0.54 g) were added to a 1 gallon Ross mixer (model DPM-4) and purged with a nitrogen blanket. The reaction medium was agitated at 30 rpm and the temperature of the reactor was raised to 60° C. A white precipitate of elastomer material in accordance with the invention formed within 15 to 20 minutes. The reactor was stirred for an additional 3 hours. The stirring was discontinued and cool to room temperature. The material was allowed to settle from the solvent solution. The supernatant was decanted and the solid was isolated. The solid was removed and filtered in a Buckner funnel. The resulting cake was washed with 400 g of acetone. The cake was transferred to a glass dish and dried in the vacuum oven at 50° C. overnight. The resulting free flowing powder was white in appearance. The resulting elastomer was comprised of 6.57 wt % phosphate moiety.

### Synthetic Example 6

Acetone (252.7 g), acrylic acid (32.1 g), 2-hydroxyethyl methacrylate phosphate (2.0 g), 2-hydroxyethyl methacrylate (5.2 g), glycerol, 1,3-dimethacrylate (0.8 g), potassium carbonate (12.5 g) and azobisisobutyronitrile (AIBN) (0.27 g) were added to a 1 L four neck flask and purged with nitrogen. The reaction medium was agitated at 200 RPM and the temperature of the reactor was raised to 55° C. A white precipitate of elastomer material in accordance with the invention formed within 15 to 20 minutes. The reactor was stirred for an additional 4-5 hours. The stirring was discontinued and the contents were cooled to room temperature. The precipitate was allowed to settle from the solution. The supernatant was decanted and the solid was isolated. The solid was removed and filtered in a Buckner funnel. The resulting cake was washed with 200 g of acetone. The cake was transferred to a glass dish and dried in a vacuum oven at 50° C. overnight. The resulting free flowing powder was white in appearance. The resulting elastomer was comprised of 0.04 wt % phosphate moiety.

### Synthetic Example 7

Water (313.7 g), acrylic acid (50.5 g) and 2-hydroxyethyl methacrylate phosphate (25.5 g) were added to a 1 L ross-mixer reactor and purged with nitrogen. The reaction was heated to 55° C. and then 1.3 g of sodium bisulfite solution (10 wt % in water) and 2.5 g ferrous ammonium sulfate solution (0.2 wt % in water) added and mixed for three minutes. After that, 15 g potassium persulfate solution (4.5 wt % in water) was added and the reaction was allowed for 2 hours while mixing. After two hours, the reaction temperature was raised to 72° C. and then 0.65 g of sodium bisulfite solution (10 wt % in water) and 1.25 g ferrous ammonium sulfate solution (0.2 wt % in water) were added and mixed for 3 minutes. 7.5 g potassium persulfate solution (4.5 wt % in water) was then added and the reaction was allowed for 1 hour while mixing. The reaction mixture cooled to 30° C. and added 30 g solution of sodium bisulfite solution (10 wt % in water) and mixed for 10 minutes. After that, 12.9 g of potassium carbonate was charged and the contents were mixed for two hours. The product was isolated as a gel in 22 wt % solid contents. This gel was dried and grinded to a fine powder and was white in appearance. The resulting elastomer was comprised of 3.56 wt % phosphate moiety.

### APPLICATION EXAMPLES

### Method of Preparation of Application Examples 1-5

Application examples 1-5, below, were prepared by mixing the solid material from Synthesis Examples 1-5 in water. The solution was mixed in a speed mixer for 3 minutes at 3000 rpm. Solutions were allowed to rest for 24 hours before testing. Similar mixing protocols were followed for the comparative examples to follow.

Testing Methods

The application examples and comparative examples were tested for 1) film formation, 2) tensioning effect, 3) whitening, 4) wrinkle hiding ability and 5) optical effect.

1) Film Formation. Film properties were tested by printing films of the formulations on a clear transparency, using a 4 inch square applicator. Wet film thickness was maintained to be 125 microns. The films were allowed to dry for 24 hours. Film properties were visualized and recorded as whether a continuous film was formed or not and whether the film adhered to the substrate or not.

2) Tensioning effect was measured by measuring the “lift distance (L)” which is defined as the vertically lifted distance (mm) of the ends of a leather substrate from its base horizontal position after treatment with the formulations. The leather substrate was a 1 mm thick white leather Style Whole Hide obtained from Testfabrics Inc. The leather was cut into 4×1 cm pieces. The leather pieces were placed on a glass substrate and 100 mg of test formulations were dotted uniformly. The solutions were spread uniformly to cover the entire surface. The coated substrates were dried at 45° C. for 3 hrs. The lift distance was measured from pictures of the curled leather pieces using image processing software (ImageJ) as the averaged vertically lifted distance of the two ends of leather from its base and was measured in replicates of 3.

3) Elastic recovery of the films was measured by placing a glass slide on the curled leather pieces from step 2 above and applying a 500 g weight for 10 seconds. After removing the weight, the leather piece was allowed to relax for 10 minutes. The lift distance L was measured as described above. % recovery was calculated by ratio of L before and after weight application.

4) Whitening was measured by using a 50 mm dia. BioSkin disk (Disc Shape BSP No. 10B, Beaulax Co., Ltd.) as a substrate. 90 mg of test solution is applied to substrate and is spread uniformly. Applied solution is allowed to dry for 1 hr. at 45 C. Whiteness and brittleness is measured by rubbing a finger over the dry film for 10 seconds with 100 gm pressure. More brittle films tend to flake while rubbed. This corresponds to a whitening effect. Whitening was observed by stereoscope. Whitening was quantified by measuring L*, a and b color values by Coloreye 7000A of the neat substrate (before) and after the test (after). AE (change in color) is calculated by

$$\sqrt{{( {L}_{before}^{*} - {L}_{after}^{*} )}^{2} + {( {a}_{before} - {a}_{after} )}^{2} + {( {b}_{before} - {b}_{after} )}^{2}} .$$

A higher ΔE value represents higher whitening effect.

5) Wrinkle hiding ability was observed by Visioscan VC 98 (Courage —Khazaka Electronic). A test solution was applied to the forearm of a consumer. Approximately 10 mg test solution was applied to a 4 cm² area of the forearm. Wrinkle hiding ability was observed by Visioscan VC 98 before application of the test solution and after 15 minutes of the solution application. 200 micron thick films of 5% solutions were printed on VitroSkin. After drying, direct transmission and diffused transmission was measured using color-eye 7000A.

### APPLICATION EXAMPLES

| | |
|---|---|
| Application Example 1 | 5% solution of Synthesis Example 1 in water |
| Application Example 2 | 5% solution of Synthesis Example 2 in water |
| Application Example 3 | 5% solution of Synthesis Example 3 in water |
| Application Example 4 | 5% solution of Synthesis Example 4 in water |
| Application Example 5 | 5% solution of Synthesis Example 5 in water |

### COMPARATIVE EXAMPLES

| | |
|---|---|
| Comparative Example 1 | 5% solution of sodium silicate in water |
| Comparative Example 2 | 5% solution of Acrylate cross-linked silicone |
| | copolymer |
| Comparative Example 3 | 5% solution of Covacryl-40¹ |
| Comparative Example 4 | 5% solution of Covacryl-60¹ |
| Comparative Example 5 | 5% solution of crosslinked sodium acrylate |

¹Sodium Polyacrylate, from Sensient Cosmetic Technologies

Results

1) Film Properties—

Table I lists film properties of the Application Examples and the Comparative Examples

TABLE I

| | | Adherence to |
|---|---|---|
| | Continuous film | substrate |
| Test solution | (yes/no) | (yes/no) |
| Application example 1 | Yes | Yes |
| Application example 2 | Yes | Yes |
| Application example 3 | Yes | Yes |
| Application example 4 | Yes | Yes |
| Application example 5 | Yes | Yes |
| Comparative Example 1 | Yes | Yes |
| Comparative Example 2 | No | Yes |
| Comparative Example 3 | Yes | No |
| Comparative Example 4 | Yes | No |
| Comparative Example 5 | Yes | No |

The Application Examples, in accordance with preferred embodiments of the invention, all formed continuous films that adhered to the substrate. Other than Comparative Example 1, the Comparative Examples either formed films that did not adhere to the substrate or tended to leave dried powder on the substrate, rather than forming a continuous film.

Tensioning Effect—

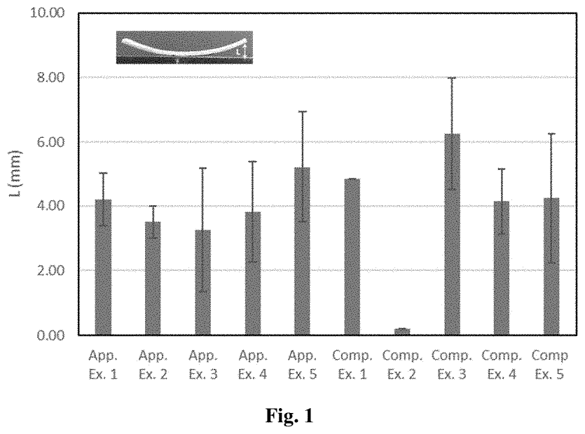

Table II and FIG. 1 shows the lift distance (L) of test solutions.

TABLE II

| | L |
|---|---|
| | (mm) |
| App. Ex. 1 | 4.2 |
| App. Ex. 2 | 3.5 |
| App. Ex. 3 | 3.3 |
| App. Ex. 4 | 3.8 |
| App. Ex. 5 | 5.2 |
| Comp. Ex. 1 | 4.9 |
| Comp. Ex. 2 | 0.2 |
| Comp. Ex. 3 | 6.3 |
| Comp. Ex. 4 | 4.2 |
| Comp. Ex. 5 | 4.2 |

As seen in Table II and FIG. 1, except for Comparative Example 2, all the Examples showed a tensioning effect as measured by a curling of the substrate.

Elastic Recovery—

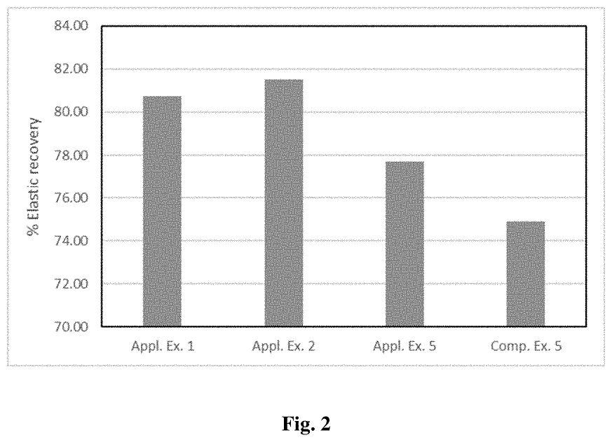

The Application Examples show a higher % elastic recovery compared to Comparison Example 5, as shown in Table III and FIG. 2.

TABLE III

| | % |
|---|---|
| | Elastic |
| | recovery |
| App. Ex. 1 | 81 |
| App. Ex. 2 | 82 |
| App. Ex. 5 | 78 |
| Comp. Ex. 5 | 74 |

Whitening Effect—

Stereoscope images of the BioSkin before and after rubbing.

Films of the Comparative Examples showed brittleness observed as white flakes on the substrate. The brittleness leading to flakiness results in whiteness on the substrate.

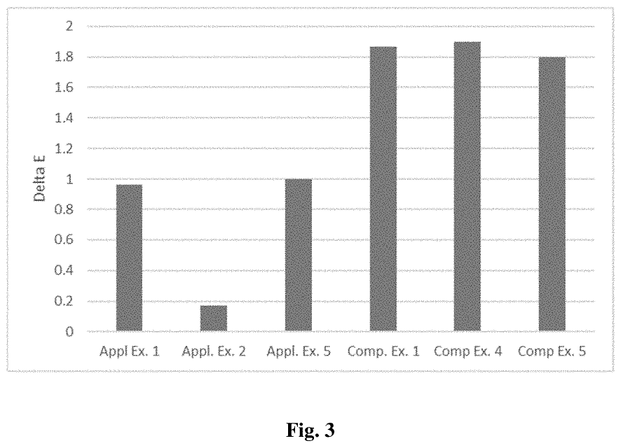

Table IV and FIG. 3 shows whiteness as measured as ΔE, after rubbing over the dried film. Higher ΔE indicates higher whitening, caused by flakes of a brittle film.

TABLE IV

| | ΔE |
|---|---|
| App. Ex. 1 | 0.96 |
| App. Ex. 2 | 0.17 |
| App. Ex. 5 | 1.00 |
| Comp. Ex. 1 | 1.87 |
| Comp. Ex. 4 | 1.90 |
| Comp. Ex. 5 | 1.79 |

Wrinkle Hiding Ability—

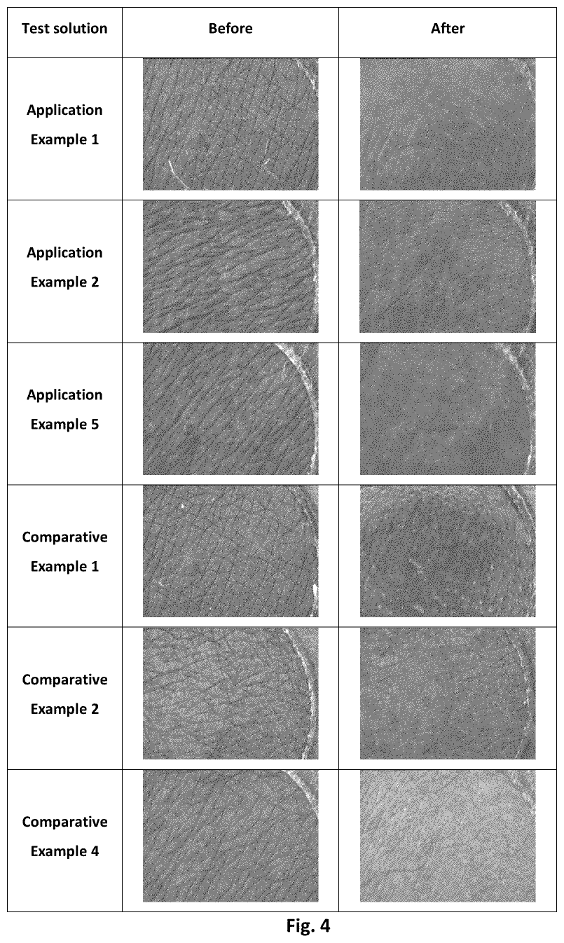

FIG. 4 shows Visioscan images of the forearm of a test subject, before and after drying the test solutions on the subject's forearm for 15 minutes, Application Examples of present invention smoothened wrinkled leading to their masking.

Optical Effects—

Optical effects, in terms of light scattering, are quantified by measuring diffused transmission of dried films of solutions. Table V shows % diffused transmission of dried films.

TABLE V

| | % Diffused |
|---|---|
| | transmission |
| Blank VitroSkin | 76.95 |
| Appl. Ex. 1 | 97.26 |
| Appl. Ex. 2 | 97.44 |
| Appl. Ex. 3 | 97.26 |
| Appl. Ex. 4 | 97.38 |
| Appl. Ex. 5 | 97.22 |

As seen in above Table V, the examples of the invention increased the diffused transmission after drying. Increased diffused transmission will enhance optical scattering to blur skin imperfections

The above results show that the Application Examples of the present invention improve skin tightening, skin smoothness and temporarily hide wrinkles. In addition, they provide silky sensory feel and enhanced optical properties The results also show that compositions in accordance with the invention exhibit less whitening than the comparative solutions.

The water swellable polyacrylate described herein (e.g., Synthesis Examples 1-5) can be used in broad range of personal care formulations, including skin care, color cosmetics and sun care formulations such as eye cream, moisturizers, night repair lotions, sun creams, lipsticks, foundations, lotions, to provide skin tightening and wrinkle smoothening benefits.

### Application Example 6

A refreshing melting gel of the following composition (Table VI) was made by charging a container with water phase A. The ingredients of phase A were mixed and then homogenized until a uniform gel was formed. All the ingredients of oil phase B were mixed separately. Then, phase B was added to phase A with intensive stirring. The formulation was homogenized for 1-2 min (Ultra Turrax-9500 rpm). Finally, the ingredients of part C were added and mixed until uniform.

TABLE VI
Refreshing melting gel

| | Ingredient | INCI | Wt. % |
|---|---|---|---|
| A | Water | Aqua | Up to |
| | | | 100.0 |
| | Sepinov EMT 10^{a} | Hydroxyethyl acrylate/sodium acryloyl | 1.30 |
| | | dimethyl taurate copolymer | |
| | Synthesis | | 5.00 |
| | Example 1 | | |
| B | Cetiol E^{b} | PPG-15 Stearyl Ether | 2.00 |
| | Cetiol 868^{b} | Ethylhexyl Stearate | 3.00 |
| | Argan oil | Argan oil | 1.00 |
| | SF 1202^{c} | Cyclopentasiloxane | 3.00 |
| | Silsoft* 034 fluid^{c} | Caprylyl Methicone | 2.00 |
| C | Silsoft E-Pearl | Water (and) Polymethylsilsesquioxane | 2.00 |
| | PMF emulsion^{c} | (and) Dimethicone (and) | |
| | | Isohexadecane (and) Cetearyl | |
| | | Methicone (and) PEG-40 Stearate | |
| | | (and) Steareth-2 (and) | |
| | | Steareth-21 | |
| | Preservative | | 0.1 |
| | Fragrance | Fragrance | 0.1 |

^{a}from Seppic
^{b}from BASF
^{c}from Momentive Performance Materials
*Silsoft is a trademark of Momentive Performance Materials.

### Application Example 7

All the ingredients (see Table VII) of oil phase B were mixed and heated to 70° C. Separately, xanthan Gum was dispersed in Glycerin and mixed in water. Synthesis Example 1 was added and mixed. Water phase A was heated to 70° C. Oil phase B was added to phase A with intensive stirring. The combination was homogenized for 1-2 min (UltraTurrax-9500 rpm). Ingredients of phase C were added when the mixture had cooled to 50° C. The mixture was cooled to room temperature and the pH adjusted to 7.

TABLE VII
Silky Eye Gel

| | | | Wt. |
|---|---|---|---|
| | Ingredient | INCI | % |
| A | Water | Aqua | Up to |
| | | | 100.0 |
| | Keltrol CG-SFT^{a} | Xanthan Gum | 0.07 |
| | Synthesis | | 5.00 |
| | Example 2 | | |
| | Glycerin | Glycerin | 2.50 |
| | Disodium EDTA | Disodium EDTA | 0.05 |
| B | Biophilic H^{b} | Hydrogenated Lecithin, | 2.30 |
| | | C12-C16 Alcohols, Palmitic Acid | |
| | Heliogel^{b} | Sodium Acrylates Copolymer, | 1.50 |
| | | Hydrogenated Polyisobutene, | |
| | | Phospholipids, | |
| | | Polyglyceryl-10 Stearate, Helianthus | |
| | | Annuus Seed Oil | |
| | Lanette 16^{c} | Cetyl Alcohol | 1.00 |
| | Crodamol MM^{d} | Myristyl Myristate | 1.20 |
| | Arlamol HD^{d} | Isohexadecane | 1.50 |
| | Miglycol 8810^{e} | Butylene Glycol Dicaprylate/Dicaprate | 1.00 |
| | Cetiol CC^{c} | Dicaprylyl Carbonate | 2.00 |
| | Pripure 3759^{d} | Squalane | 0.50 |
| | Arlamol PS15E-LQ^{d} | PPG-15 Stearyl Ether | 0.50 |
| | BHT | BHT | 0.10 |
| | Tospearl* 3000A | Polymethylsilsesquioxane | 2.00 |
| | microspheres^{f} | | |
| | Velvesil* DM gel^{f} | Dimethicone (and) Cetearyl | 5.00 |
| | | Dimethicone Crosspolymer | |
| | Silsoft* 034 fluid^{f} | Caprylyl Methicone | 2.00 |
| C | Euxyl PE 9010^{g} | Phenoxyethanol, Ethylhexylglycerin | 1.00 |
| | Moist 24^{h} | Imperata cylindrica root extract (and) | 2.00 |
| | | water (and) glycerin (and) PEG-8 | |
| | | (and) carbomer | |
| | Sodium Hyaluronate | Sodium Hyaluronate | 1.00 |
| | (10%) | | |
| | Fragrance | Fragrance | 0.1 |

^{a}from CP Kelco
^{b}from Lucas Meyer Cosmetics
^{c}from BASF
^{d}from Croda
^{e}from IOI Oleo GmbH
^{f}from Momentive Performance Materials
^{g}Schulke Inc.
^{h}from Sederma
*Velvesil and Silsoft are trademarks of Momentive Performance Materials.

### Application Example 8

All the ingredients (see Table VIII) in phase A were combined in a vessel. The ingredients were mixed until homogeneous. The phase B ingredients were dissolved in water separately. The phase B ingredients were added into phase A slowly and the mixture was homogenized.

TABLE VIII
Night Cream

| | Ingredient | INCI | Wt. % |
|---|---|---|---|
| A | Silsoft* DML^{a} | Dimethicone | 14.00 |
| | SilForm* 60A^{a} | PEG/PPG-20/15 Dimethicone | 4.00 |
| | | (and) Diisopropyl Adipate | |
| | Velvesil* 034 | Caprylyl Methicone (and) C30-45 Alkyl | 5.00 |
| | gel^{a} | Cetearyl Dimethicone Crosspolymer | |
| | SF 1632^{a} | Cetearyl Methicone | 1.00 |
| | Tospearl* 3000A | Polymethylsilsesquioxane | 5.00 |
| | microspheres^{a} | | |
| B | Deionized Water | Water | Up to |
| | | | 100 |
| | Synthesis | | 2.5 |
| | Example 5 | | |
| | Zemea^{b} | Propanediol | 5.00 |
| | Glycerin | Glycerin | 1.00 |
| | Sodium Chloride | Sodium Chloride | 1.00 |
| | Germall II^{c} | Diazolidinyl Urea | 0.50 |

^{a}from Momentive Performance Materials
^{b}from DuPont Tate & Lyle Bio Products
^{d}from Ashland
*Silsoft, SilForm and Tospearl are trademarks of Momentive Performance Materials.

### Application Example 9

In a main vessel, water was mixed with EDTA. Pemulen powder was dispersed under high-speed mixing until a uniform mixture was formed. Remaining phase A ingredients (see Table IX) were added and heated to 75° C. In a separate vessel, phase B ingredients were combined and heated to 75° C. Phase B was emulsified into phase A, with high-speed mixing. Phase C ingredients were pre-mixed until trimethamine was dissolved and add to the main vessel. Phase D was added while mixing when the mixture cooled down to 40° C. Phase E was premixed until the powder is dissolved and then added to the batch at 40° C. The batch was cooled to 35° C. The remaining ingredients were added and the batch was mixed until homogenous. See Table IX.

TABLE IX
Anti-Aging Serum

| | Ingredient | INCI | Wt. % |
|---|---|---|---|
| A | Deionized Water | Water | 73.18 |
| | Disodium EDTA | Disodium EDTA | 0.15 |
| | Synthesis Example 1 | | 4.5 |
| | Pemulen TR-2 | Acrylates/C10-30 Alkyl Acrylate | 0.20 |
| | Polymer^{a} | Crosspolymer | |
| | Hydrolite-6^{b} | 1,2-Hexanediol | 2.00 |
| B | Glyceryl Stearate | Glyceryl Stearate | 0.50 |
| | Jeemate 6000 DS^{c} | PEG-150 Distearate | 0.50 |
| | Dermol 99^{d} | Isononyl Isononanoate | 4.00 |
| | Element 14 PDMS 50^{e} | Dimethicone | 3.00 |
| | Velvesil* 034 gel^{e} | Caprylyl Methicone (and) C30-45 Alkyl | 5.00 |
| | | Cetearyl Dimethicone Crosspolymer | |
| C | Deionized Water | Water | 1.00 |
| | Tris Amino | Tromethamine | 0.22 |
| D | Silsoft* Spread Max | Methoxy PEG-8 Dimethylsilylethyl | 1.00 |
| | fluid^{e} | Trimethylsilane (and) Trideceth-5 (and) | |
| | | Meroxapol 172 | |
| | Softouch* CC6004 | Boron Nitride | 5.00 |
| | boron nitride^{e} | | |
| E | Deionized Water | Water | 1.00 |
| | Germall II^{f} | Diazolidinyl Urea | 0.50 |
| F | Syn-TC^{g} | Tetradecyl Aminobutyroyylvalylaminobutyric | 2.50 |
| | | Urea Trifluoroacetate (and) Palmitoyl | |
| | | Tripeptide-5 (and) Palmitoyl Dipeptide-5 | |
| | | Diaminobutyroyl Hydroxybutyrate (and) | |
| | | Glycerin (and) Water (and) Magnesium | |
| | | Chloride | |
| | Fragrance | Fragrance | 0.05 |

^{a}from Lubrizol
^{b}from Symrise
^{c}from Jeen International
^{d}from Alzo International Inc
^{e}from Momentive Performance Materials
^{f}from Ashland
^{g}from DSM Nutritional Products, LLC
*Velvesil, Silsoft and Softouch are trademark of Momentive Performance Materials.

### Application Example 10

In a main vessel, mix phase A until homogeneous mixture is obtained. In a separate vessel mix phase B until homogeneous paste is obtained. Add phase B into phase A while continuous mixing. Homogenize the solution. Add phase C. See Table X.

TABLE X

| | | | |
|---|---|---|---|
| A | Sepigel 305^{a} | Polyacrylamide, C13-14 Isoparaffin, | 1.5 |
| | | Laureth-7 | |
| | Aqua | Aqua | q.s. 100 |
| | SilForm* HyFlex Emulsion^{b} | Bis-Ethylhydroxycyclohexyl | 8.0 |
| | | Methacrylate Dimethicone/Butyl | |
| | | Acrylate Crosspolymer | |
| | Synthesis Example 2 | | 4.0 |
| B | SF1202^{b} | Cyclopentasiloxane | 7.0 |
| | Silsoft* 034^{b} | Caprylyl Methicone | 5.0 |
| | MT-100TV Titanium dioxide^{c} | Titanium Dioxide | 5.0 |
| | SunPURO Red Iron Oxide^{d} | Iron Oxides Red | 0.3 |
| | SunPURO Black Iron Oxide^{d} | Iron Oxides Black | 0.1 |
| | SunPURO Yellow Iron Oxide^{d} | Iron Oxides Yellow | 0.9 |
| | Bentone gel VS-5 PC V^{d} | Cyclopentasiloxane (and) | 3.0 |
| | | Disteardimonium Chloride (and) | |
| | | Propylene Carbonate | |
| C | Glydant Plus^{e} | DmDm Hydantoin | 0.5 |

^{a}from Seppic
^{b}from Momentive Performance Materials
^{c}from Tayca corporation
^{d}from Sun Chemical
^{e}from Lonza
*SilForm and Silsoft are trademarks of Momentive Performance Materials.

As shown herein, preferred elastomers, as siloxane polymers with acrylic or methacrylic and phosphate functionality, can be formulated into film forming cosmetic products that can reduce the appearance of fine lines and wrinkles and without whitening caused by flaking. A personal care composition forming a film on a keratinous substrate can be provided. The composition can exhibit tightening of the keratinous substrate upon drying. The personal care composition can lead to smoothening of keratinous substrate. This can mask skin wrinkles temporarily. A personal care composition in accordance with the invention can optionally be free of oils. The personal care application can be selected from the group of but are not limited to eye creams and lotions, deodorants, antiperspirants, antiperspirant/deodorants, shaving products, skin lotions, moisturizers, toners, bath products, cleansing products, hair care products, manicure products, protective creams, color cosmetics and other personal care formulations where silicone components have been conventionally added, as well as drug delivery systems for topical application of medicinal compositions that are to be applied to the skin. The personal care application can further comprise at least one personal care ingredient selected from the group consisting of emollients, moisturizers, humectants, pigments, colorants, fragrances, biocides, preservatives, antioxidants, anti-microbial agents, anti-fungal agents, antiperspirant agents, exfoliants, hormones, enzymes, medicinal compounds, vitamins, salts, electrolytes, alcohols, polyols, absorbing agents for ultraviolet radiation, botanical extracts, surfactants, silicone oils, volatile silicones, organic oils, waxes, film formers, and thickening agents.

1. A cosmetically acceptable composition, comprising a water swellable cross linked polyacrylate elastomer containing at least one phosphate or phosphonate moiety, the elastomer formulated and adapted such that after the composition has been applied to a keratinous substrate in a water swelled condition, upon drying from the water swelled condition, the elastomer adheres to the substrate, shrinks and as it shrinks, provides tightening and smoothening of the substrate to which the composition had been applied.

2. The cosmetically acceptable composition of claim 1, wherein the elastomer contains a diradical moiety of phosphate or phosphonate, having the general formula (I): 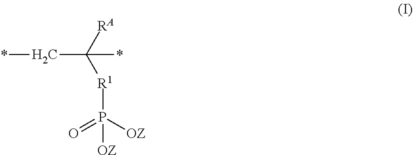
wherein R¹ is a C1-C60 alkyl chain which can be linear, branched or cyclic and optionally containing a heteroatom (O, N, S); Z is H or an alkali or alkaline metal ion or an ammonium ion; * is a bond to the structure of the elastomer; and R^{A} is H or CH₃.

3. The cosmetically acceptable composition of claim 1, wherein the elastomer contains a diradical moiety of an acrylic acid, an acrylic acid salt or an ester, having the general formula (II): 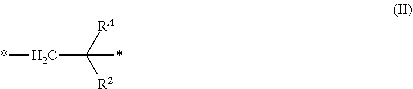
wherein R² is a C1-C60 alkyl chain, which can be linear, branched or cyclic optionally containing a heteroatom (O, N, S); * is a bond to the structure of the elastomer; and R^{A} is H or CH₃.

4. The cosmetically acceptable composition of claim 1, wherein the elastomer comprises a diradical moiety having the general formula III or IV: 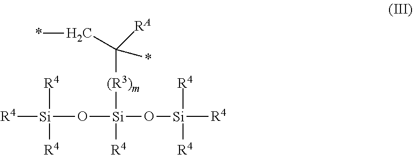
wherein m is 0 or 1; R³ is a C1-C60 alkyl chain which can be linear, branched or cyclic optionally containing a heteroatom (O, N, S); and each R⁴ is independently a monovalent hydrocarbon radical independently having from 1 to 20 carbon atoms; * is a bond to the structure of the elastomer; n=3 to 500; and R^{A} is H or CH₃; or 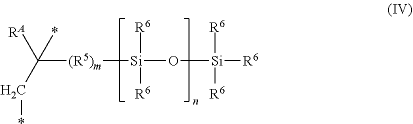
wherein m is 0 or 1; R⁵ is a C1-C60 alkyl chain which can be linear, branched or cyclic optionally containing a heteroatom (O, N, S); R^{A} is H or CH₃; * is a bond to the structure of the elastomer; n=3 to 500; and each R⁶ is independently a monovalent hydrocarbon radical having from 1 to 20 carbon atoms.

5. The cosmetically acceptable composition of claim 1, wherein the elastomer includes about 0.01 wt %-40 wt % of the phosphate moiety.

6. The cosmetically acceptable composition of claim 1, wherein the elastomer includes about 2 wt %-15 wt % of the phosphate moiety.

7. The water swellable elastomer of claim 1, wherein the elastomer comprises moieties having the general formulae (I), (II), (III) or (IV).

8. The cosmetically acceptable composition of claim 1, wherein when undisturbed, at least about 75 wt % of the elastomer film will remain as a film on the substrate, without forming flakes, after six hours.

9. The cosmetically acceptable composition of claim 1, wherein the tightening applied to the keratinous substrate urges a smoothening of the keratinous substrate.

10. The cosmetically acceptable composition of claim 1, in an amount effective to temporarily reduce the appearance of skin wrinkles of a user to which the composition is applied.

11. The cosmetically acceptable composition of claim 1 wherein the composition is formulated as a personal care product selected from the group consisting of eye creams and lotions, skin firming creams and lotions, antiaging lotions and creams, facial cleansers, toners, deodorants, antiperspirants, shaving products, skin lotions, moisturizers, bath products, cleansing products, hair care products, manicure products, protective creams, facial masks, skin care products or color cosmetics.

12. The cosmetically acceptable composition of claim 1, further comprising a thickener, a particulate, a plasticizer, an emulsifier or a skin care active agent.

13. A method of reducing the appearance of wrinkles for at least four hours, consisting essentially of applying the composition of claim 1 to skin having wrinkles.

14. A water swellable elastomer formed as the reaction product of reactant components, comprising:
an acrylate or methacrylate moiety containing component having 3 to 60 carbon atoms;
a phosphate or phosphonate moiety containing component having 3 to 60 carbon atoms;
a siloxane component having a siloxane moiety; and
wherein the water swellable elastomer is formulated and adapted such that after the elastomer has been applied to a keratinous substrate in a water swelled condition, upon drying from the water swelled condition, the elastomer forms a film and adheres to the substrate, shrinks and as it shrinks, provides tightening and smoothening of the substrate to which the composition had been applied and the acrylate or methacrylate component, phosphate or phosphonate component and siloxane component may be the same or different components and comprise an unsaturated straight, branched or cyclical carbon chain, which may optionally include heteroatoms.

15. The water swellable elastomer of claim 14, wherein the reactant components further comprise a monomer component having 3 to 60 carbon atoms.

16. The water swellable elastomer of claim 14, wherein at least one of the reactants comprises a monomer with the general formula (V): 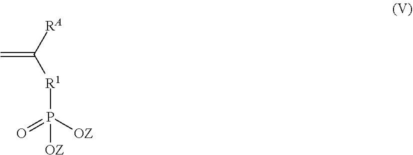
wherein R¹ is a C1-C60 alkyl chain, which can be linear, branched or cyclic optionally containing a heteroatom (O, N, S); Z is H or an alkali or alkaline metal ion or an ammonium ion; and R^{A} is H or CH₃.

17. The water swellable elastomer of claim 14, wherein at least one of the reactants comprises a monomer with the general formula (VI): 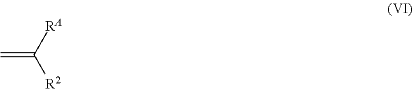
wherein R² is a C1-C60 alkyl chain which can be linear, branched or cyclic and optionally containing a heteroatom (O, N, S); and R^{A} is H or CH₃.

18. The water swellable elastomer of claim 14, wherein at least one of the reactants has general formula VII or VIII: 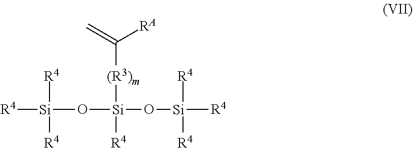
wherein m is 0 or 1; R³ is a C1-C60 alkyl chain which can be linear, branched or cyclic optionally containing a heteroatom (O, N, S); each R⁴ is independently a monovalent hydrocarbon radical independently having from 1 to 20 carbon atoms; n=3 to 500; and R^{A} is H or CH₃; or 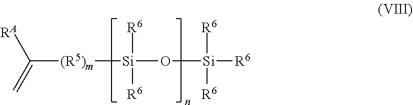
wherein m is 0 or 1; R⁵ is a C1-C60 alkyl chain which can be linear, branched or cyclic optionally containing a heteroatom (O, N, S); each R6 is independently a monovalent hydrocarbon radical having from 1 to 20 carbon atoms; n=3 to 500; and R^{A} is H or CH₃.

19. The cosmetically acceptable composition of claim 14, wherein at least one of the reactants is a cross-linker of general formula (IX)
R⁷(R⁸)ₐ (IX)
wherein a≥2;
R⁷ is a bivalent group connected to R⁸ and R⁷ can be
i) a bivalent hydrocarbon radical having from 1-20 carbon atoms, optionally containing heteroatom (O, S, N);
ii) a bivalent phosphoric acid formed by the bonding of O and an alkyl radical;
iii) [R⁹R¹⁰SiO]ₙ wherein R⁹ and R¹⁰ are monovalent hydrocarbon radicals having from 1 to 20 carbon atoms, n=3-500; or
iv) MᵤDᵥTₓQ_{y} (M=R¹¹₃SiO_{1/2}, D=R¹²₂SiO_{2/2}, T=R¹³SiO_{3/2}, Q=SiO_{4/2}) wherein R¹¹, R¹², R¹³ are monovalent hydrocarbon radicals having from 1 to 20 carbon atoms; u, v, x, y are independent integers from 0 to 500, where u+v+x+y≥1; and
R⁸ is a monovalent unsaturated hydrocarbon radical connected to R⁷.

20. The water swellable elastomer of claim 14, wherein the elastomer comprises the reaction product of a vinyl terminated polydimethylsiloxane and an acrylic or methacrylic siloxane copolymer.

21. The water swellable elastomer of claim 14, wherein at least one of the reactants is 2-hydroxyethyl methacrylate phosphate.

22. The water swellable elastomer of claim 14, wherein at least one of the reactants is a vinyl terminated polydimethylsiloxane with the average structure CH₂—CHSi(CH₃)₂O[Si(CH₃)₂O]ₙSi(CH₃)₂CH═CH₂, wherein n is about 20 to 2000.

23. The water swellable elastomer of claim 14, wherein at least one of the reactants comprises tripropyleneglycolmethacrylate monophosphate.

24. The water swellable elastomer of claim 14, wherein the elastomer is formed as a polymer of a plurality of polydimethyl siloxane monomer units comprising about 100 to 2000 siloxane groups.

25. The water swellable elastomer of claim 14, wherein about 2 wt %-50 wt % of the reactant components comprise a phosphate or phosphonate monomer.

26. The water swellable elastomer of claim 14, wherein about 20 wt %-40 wt % of the reactant components comprise a phosphate or phosphonate monomer.

27. The water swellable elastomer of claim 14, formulated and adapted so that when in a water condition, the elastomer is applied to a keratinaceous substrate and left to dry to a film, undisturbed, at least about 75 wt % of the elastomer will remain as a continuous film adhered to the substrate, without flaking after six hours.

28. The water swellable elastomer of claim 14, in an amount effective to temporarily reduce the appearance of skin wrinkles of a user in need thereof at a location where the elastomer is applied.

29. A method of reducing the appearance of wrinkles in wrinkled skin, consisting essentially of applying the elastomer of claim 14 to the wrinkled skin in the water swollen condition and permitting the swollen elastomer to dry.

30. A method of forming a water swellable elastomer, comprising combining:
an acrylate or methacrylate moiety containing monomer having 3 to 60 carbon atoms;
a phosphate moiety containing component having 3 to 60 carbon atoms;
a siloxane component; and
wherein the elastomer is formulated to form a film adhered to a keratinaceous substrate and then shrink as it dries, while remaining adhered to the substrate, and the acrylate component, phosphate component and siloxane component may be the same or different components and comprise an unsaturated straight, branched or cyclical carbon chain, which may optionally include heteroatoms.
